(19) United States
(12) Patent Application Publication
(10) Pub. No.: US 2022/0151884 A1
(43) Pub. Date: May 19, 2022

(54) COSMETIC EMULSION FOR SELF-TANNING

(71) Applicant: Shiseido Company, Ltd., Chuo-ku, Tokyo (JP)

(72) Inventor: Daiki ARAI, Tokyo (JP)

(73) Assignee: Shiseido Company, Ltd., Chuo-ku, Tokyo (JP)

(21) Appl. No.: 17/435,926

(22) PCT Filed: Feb. 19, 2020
(86) PCT No.: PCT/JP2020/006629

§ 371 Date: Sep. 2, 2021

(30) Foreign Application Priority Data
Mar. 4, 2019 (JP) 2019-038236

(51) Int. Cl. A61K 8/06; A61K 8/35; A61K 8/81; A61K 8/86; A61Q 19/04

(52) U.S. Cl. CPC A61K 8/06; A61K 8/35; A61K 8/8158; A61K 2800/654; A61K 8/86; A61Q 19/04; A61K 2800/412; A61K 8/8152

(57) ABSTRACT

[Problem] An objective of the present invention is to provide an emulsion cosmetic for self-tanning, which has an excellent texture to the touch, which exhibits strong resistance to contact (rubbing) with water, clothes, fingers and the like, and which does not tend to result in uneven coloring. [Solution] The self-tanning emulsion cosmetic according to the present invention comprises (A) core-corona microparticles in which hydrophilic groups are partially provided on surfaces of hydrophobic fine particles; and (B) a self-tanning agent.

### TECHNICAL FIELD

The present invention relates to an emulsion cosmetic for self-tanning, which is resistant to removal even when coming into contact with water, clothes, fingers or the like, and which can color skin evenly.

### BACKGROUND ART

Self-tanning is also called sunless tanning, and refers to applying a cosmetic containing a self-tanning agent to skin, thereby creating brown skin similar to a suntan without exposure to sunlight. Self-tanning agents change skin color as a result of reacting with amino acids in the stratum corneum of the skin, and dihydroxyacetone (DHA) and the like are generally known. Self-tanning is favored for being able to make skin appear healthy without the skin being affected by harmful ultraviolet rays.

In order to obtain beautiful brown skin by self-tanning, the cosmetic must be applied uniformly, without unevenness. Additionally, cosmetics with a good texture to the touch are sought because they are applied and left for a certain period of time.

For example, Patent Document 1 proposes a gel-type self-tanning cosmetic characterized by containing dihydroxyacetone, water, alcohol, a cellulose-based water-soluble thickener and/or xanthan gum, and a chelating agent. In this gel-type self-tanning cosmetic, a lotion-type formulation is thickened to increase the viscosity, thereby obtaining a good texture to the touch and eliminating dripping at the time of application and unevenness on the skin.

Additionally, Patent Document 2 proposes an emulsified gel composition characterized by containing dihydroxyacetone and a thickener consisting of a microgel obtained by dissolving a water-soluble ethylenically unsaturated monomer in a dispersion phase and inducing radical polymerization in the dispersion phase. This emulsified gel composition achieves excellent thickening effects by means of the microgel, even in a composition having a high ethanol content, thus providing a good, fresh feeling in use and achieving excellent base stability.

However, cosmetics that are applied to skin can partially run off due to water from the outside environment or from perspiration secreted from the skin, or can be removed by coming into contact with clothes or fingers. Conventional self-tanning cosmetics could not be considered to be fully satisfactory in terms of maintaining a coating film after being applied and spread on the skin.

### RELATED ART

### Patent Documents

Patent Document 1: JP H7-101848 A

Patent Document 2: JP 2005-145860 A

### SUMMARY OF THE INVENTION

### Problem to be Solved by the Invention

An object of the present invention is to provide an emulsion cosmetic for self-tanning, which has an excellent texture to the touch, which exhibits strong resistance to contact (rubbing) with water, clothes, fingers and the like, and which does not tend to result in uneven coloring.

### Means for Solving the Problem

As a result of diligent investigation towards solving the aforementioned problems, the present inventors discovered, surprisingly, that a coating film is made more strongly resistant to rubbing by clothes, fingers or the like by using core-corona microparticles as sn emulsifying agent in a self-tanning emulsion cosmetic, thereby completing the present invention.

In other words, the present invention provides an emulsion cosmetic for self-tanning, comprising:

(A) core-corona microparticles in which hydrophilic groups are partially provided on surfaces of hydrophobic fine particles; and
(B) a self-tanning agent.

### Effects of the Invention

By having the above-mentioned features, the present invention exhibits strong resistance to contact (rubbing) with water, clothes, fingers or the like, and the coating film is resistant to removal, thereby allowing skin to be evenly colored. Since core-corona microparticles are used as an emulsifying agent, stickiness can be suppressed and wateriness can be imparted in comparison with emulsification methods using surfactants. Furthermore, since the hydrophobic fine particles that are the core particles are softer than inorganic fine particles, a powdery feeling in use can be reduced more than in the case of Pickering emulsion methods using inorganic fine particles.

### MODES FOR CARRYING OUT THE INVENTION

As mentioned above, the cosmetic of the present invention comprises (A) core-corona microparticles in which hydrophilic groups are partially provided on surfaces of hydrophobic fine particles; and (B) a self-tanning agent. Hereinafter, the ingredients constituting the cosmetic of the present invention will be explained in detail.

### <(A) Core-Corona Microparticles>

In the present invention, the (A) core-corona microparticles (hereinafter, sometimes referred to simply as “component (A)”) may be crosslinked or non-crosslinked core-corona microparticles in which hydrophilic groups are partially provided on surfaces of hydrophobic fine particles.

Examples of particularly preferred core-corona microparticles include (acrylates/methoxy PEG methacrylate) crosspolymer [crosslinked core-corona microparticles] and acrylamide-based core-corona microparticles such as (acrylamide/DMAPA acrylate/methoxy PEG methacrylate) copolymer [non-crosslinked core-corona microparticles], as indicated below.

### 1. Crosslinked Core-Corona Microparticles

The crosslinked core-corona microparticles according to the present invention can be obtained by radical polymerization, under specific conditions, of the monomers indicated by Formulas (1) to (3) below. As an example, there is (acrylates/methoxy PEG-90 methacrylate) crosspolymer.

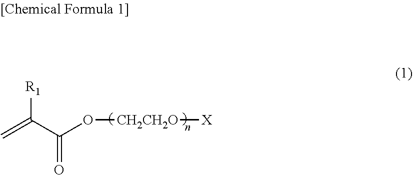

In Formula (1), R₁ is an alkyl group having 1 to 3 carbon atoms, and n is a number from 8 to 200. X is H or CH₃.

The polyethylene oxide macromonomer indicated by the above Formula (1) may, for example, be a commercially available product sold by Aldrich, or a commercially available product such as Blemmer (registered trademark), sold by NOF.

The molecular weight (i.e., the value of n) of the polyethylene oxide moiety must be n=8 to 200.

Thus, the macromonomer may, for example, be Blemmer (registered trademark) PME-400, Blemmer (registered trademark) PME-1000, Blemmer (registered trademark) PME-4000 or the like, manufactured by NOF.

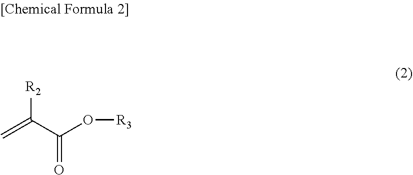

In Formula (2), R₂ is an alkyl group having 1 to 3 carbon atoms. R₃ is an alkyl group having 1 to 12 carbon atoms, more preferably an alkyl group having 1 to 8 carbon atoms.

The hydrophobic monomer indicated by Formula (2) above may be a commercial product that is sold, for example, by Aldrich or Tokyo Chemical Industry.

The hydrophobic monomer may, for example, be methyl acrylate, ethyl acrylate, propyl acrylate, butyl acrylate, pentyl acrylate, hexyl acrylate, heptyl acrylate, octyl acrylate, decyl acrylate, dodecyl acrylate, methyl methacrylate, ethyl methacrylate, propyl methacrylate, butyl methacrylate, pentyl methacrylate, hexyl methacrylate, heptyl methacrylate, octyl methacrylate, decyl methacrylate, dodecyl methacrylate or the like. In particular, it is preferable to use methyl methacrylate, butyl methacrylate or octyl methacrylate.

These hydrophobic monomers are general-purpose raw materials and are also easily available as general industrial raw materials.

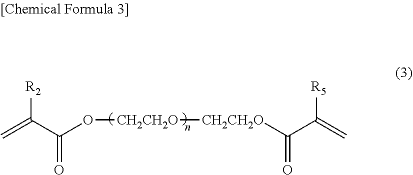

In Formula (3), R₄ and R₅ each independently represent an alkyl group having 1 to 3 carbon atoms, and m is a number from 0 to 2.

The crosslinkable monomer indicated by Formula (3) above is available as a commercial product or as an industrial raw material. This crosslinkable monomer is preferably hydrophobic.

The value of m is preferably from 0 to 2. Specifically, ethylene glycol dimethacrylate (hereinafter sometimes abbreviated to EGDMA), which is sold by Aldrich, Blemmer (registered trademark) PDE-50, which is sold by NOF, and the like are preferably used.

The core-corona microparticles according to the present invention are obtained by radical polymerization of the above-mentioned monomers under the conditions (A) to (E) indicated below.

(A) The molar ratio, represented by the molar amount of the polyethylene oxide macromonomer that is added divided by the molar amount of the hydrophobic monomer that is added, is 1:10 to 1:250.
(B) The amount of the crosslinkable monomer that is added is 0.1% to 1.5% by mass relative to the amount of the hydrophobic monomer that is added.
(C) The hydrophobic monomer indicated by Formula (2) is a monomer composition containing one type or a mixture of two are more types of methacrylic acid derivatives having alkyl groups with 1 to 8 carbon atoms.
(D) The polymerization solvent is a mixed solvent containing water and an organic solvent. If a polyol is used as the organic solvent, then it should be one or more types selected from among dipropylene glycol, 1,3-butylene glycol and isoprene glycol.
(E) The solvent composition of the mixed solvent of water and the organic solvent is such that water:organic solvent=90 to 10:10 to 90, in terms of the mass ratio at 20° C.

In the present invention, the “amount of the crosslinkable monomer that is added relative to the amount of the hydrophobic monomer that is added” is defined as the crosslinking density (in percentage by mass). The crosslinking density of the core-corona microparticles used in the present invention must, due to condition (B), be such that the amount of the crosslinkable monomer that is added relative to the amount of the hydrophobic monomer that is added is 0.1% to 1.5% by mass.

### (Condition (A))

Regarding the molar amounts of the polyethylene oxide macromonomer and the hydrophobic monomer that are added, polymerization is possible when polyethylene oxide macromonomer:hydrophobic monomer=1:10 to 1:250 (molar ratio). The above-mentioned molar amounts that are added are more preferably 1:10 to 1:200, even more preferably 1:25 to 1:100.

If the molar amount of the hydrophobic monomer is less than 10 times the molar amount of the polyethylene oxide macromonomer, then the polymerized polymer becomes water-soluble and a gel is not formed by the core-corona polymer microparticles and the solvent. Additionally, if the molar amount of the hydrophobic monomer is more than 250 times the molar amount of the polyethylene oxide macromonomer, then the dispersion stabilization due to the polyethylene oxide macromonomer becomes incomplete, and hydrophobic polymers formed by the insoluble hydrophobic monomer aggregate and precipitate.

### (Condition (B))

By copolymerizing the crosslinkable monomer, microparticles in which hydrophobic polymers on the core moieties are crosslinked can be polymerized.

If the amount of the crosslinkable monomer that is added is less than 0.1% by mass of the amount of the hydrophobic monomer that is added, then the crosslinking density is low and the microparticles collapse when swollen. Additionally, if more than 1.5% by mass is added, then the microparticles aggregate with each other, and favorable microparticles having a narrow grain size distribution cannot be polymerized. The amount of the crosslinkable monomer that is added is preferably 0.2% to 1.0% by mass, more preferably 0.2% to 0.8% by mass, and most preferably 0.2% to 0.5% by mass.

### (Condition (C))

The hydrophobic monomer indicated by Formula (2) must be a monomer composition containing one type or a mixture of two or more types of methacrylic acid derivatives having alkyl groups with 1 to 8 carbon atoms. If the number of carbon atoms is 0 (if the monomer has no terminal ester bonds), then there are cases in which the monomer is too hydrophilic and good emulsion polymerization cannot be achieved. On the other hand, if the number of carbon atoms is 9 or more, then there are conformational obstacles to polymerization, and there are cases in which a crosslinked structure cannot be well constructed.

### (Condition (D))

The polymerization solvent must be a mixed solvent containing water and an organic solvent. As the organic solvent, ethanol, propanol, butanol, a polyol or the like may be used. However, when a polyol is used, it should preferably be able to dissolve the hydrophobic monomer indicated by Formula (2) and the crosslinkable monomer indicated by Formula (3). The polyol used in the present invention must be dipropylene glycol, 1,3-butylene glycol or isoprene glycol.

Considering the fact that a polymerization solution that can be industrially manufactured, i.e., without a purification step such as dialysis, is used directly as a raw material, the solvent to be mixed with water should preferably be a polyol that can generally be blended into cosmetics, rather than an organic solvent for which irritation of the skin at the time of application would be a concern, such as ethanol, propanol, butanol or the like.

### (Condition (E))

The solvent composition of the mixed solvent of water and the organic solvent, which is the polymerization solvent, should be such that water:organic solvent=90 to 10:10 to 90, in terms of the mass ratio at 20° C. The solvent composition of the mixed solvent of water and the organic solvent should preferably be such that water:organic solvent=90 to 10:10 to 90 (by volume ratio at 20° C.), and more preferably such that water:organic solvent=80 to 20:20 to 80 (by volume ratio at 20° C.).

An organic solvent must be added to the polymerization solvent in order to homogeneously dissolve the hydrophobic monomer. The mixing ratio of the organic solvent is 10 to 90 by volume. If the mixing ratio of the organic solvent is lower than 10 by volume, then the capacity to dissolve the hydrophobic monomer becomes extremely low, and polymerization progresses in the monomer droplet state, causing large lumps to be formed without producing microparticles. Additionally, if the mixing ratio of the organic solvent exceeds 90 by volume, then a hydrophobic monomer emulsion is not formed by hydrophobic interactions, emulsion polymerization does not progress, and microparticles are not obtained.

With the core-corona microparticles according to the present invention obtained by using a polyol, the polymerization solvent is a mixed solvent containing water and a polyol, and not containing ethanol. Therefore, a cosmetic that does not irritate the skin, even for users with sensitive skin, can be conveniently obtained.

As the polymerization initiator used in the polymerization system, a commercially available polymerization initiator that is normally used in water-soluble thermal radical polymerization can be used. With this polymerization system, polymerized microparticles having an extremely narrow grain size distribution can be obtained, even without particularly strictly controlling the stirring conditions.

### 2. Non-Crosslinked Core-Corona Microparticles [Acrylamide-Based Core-Corona Microparticles]

The non-crosslinked core-corona microparticles that are preferably used in the present invention can be obtained by radical polymerization, under specific conditions, of the monomers indicated by Formulas (1), (2) and (4) below. An example is an (acrylamide/DMAPA acrylate/methoxy PEG methacrylate) copolymer.

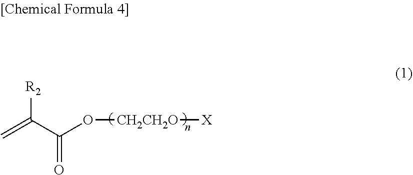

In Formula (1), R₁ is an alkyl group having 1 to 3 carbon atoms and n (the molecular weight of the polyethylene oxide moiety) is a number from 8 to 200. X is H or CH₃.

The polyethylene oxide macromonomer represented by Formula (1) above is preferably an acrylic acid derivative or a methacrylic acid derivative. For example, a commercially available product sold by Aldrich, or a commercially available product such as Blemmer (registered trademark), sold by NOF, may be used. As examples, PME-400, PME-1000 or PME-4000 (in which the values of n in Formula (1) are, respectively, n=9, n=23 and n=90, all manufactured by NOF), which are methoxy polyethylene glycol monomethalates, may be used.

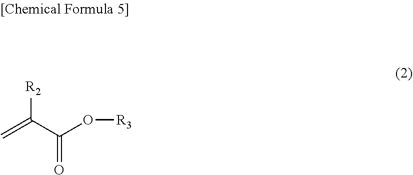

In Formula (2), R₂ represents an alkyl group having 1 to 3 carbon atoms, and R₃ represents a substituent group including an alkyl group having 1 to 12 carbon atoms.

The hydrophobic monomer represented by Formula (2) above is preferably an acrylic acid derivative or a methacrylic acid derivative, and may, for example, be methyl acrylate, ethyl acrylate, propyl acrylate, butyl acrylate, pentyl acrylate, hexyl acrylate, heptyl acrylate, octyl acrylate, decyl acrylate, dodecyl acrylate, methyl methacrylate, ethyl methacrylate, propyl methacrylate, butyl methacrylate, pentyl methacrylate, hexyl methacrylate, heptyl methacrylate, octyl methacrylate, decyl methacrylate, dodecyl methacrylate and the like. Among the above, methyl methacrylate, butyl methacrylate and octyl methacrylate are particularly preferred.

These hydrophobic monomers are general-purpose raw materials and are also easily available as general industrial raw materials. For example, commercially available products sold by Aldrich or by Tokyo Chemical Industry may be used.

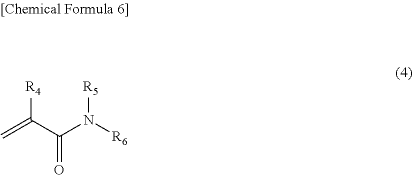

In Formula (4), R₄ represents H or an alkyl group having 1 to 3 carbon atoms, and R₅ and R₆ each independently represent H or a substituent group including an alkyl group having 1 to 18 carbon atoms.

The hydrophobic monomer represented by Formula (4) above is preferably an acrylamide derivative or a methacrylamide derivative. For example, t-butyl acrylamide, N,N-dimethylacrylamide, N-[3-(dimethylamino)propyl] acrylamide, t-butyl methacrylamide, octyl acrylamide, octyl methacrylamide, octadecyl acrylamide or the like is preferably used. Among the above, t-butyl acrylamide, N,N-dimethylacrylamide and N-[3-(dimethylamino)propyl] acrylamide are particularly preferred.

These hydrophobic monomers are available as commercial products or as industrial raw materials.

The copolymer constituting the core-corona microparticles according to the present invention is obtained by copolymerizing, by an arbitrary radical polymerization method, a macromonomer represented by Formula (1) above and one or more hydrophobic monomers selected from among those represented by Formulas (2) and (4) above, in accordance with the conditions (A) to (D) indicated below.

(A) The molar ratio, represented by the molar amount of the polyethylene oxide macromonomer that is added divided by the molar amount of the (acrylate derivative monomer and/or the acrylamide derivative monomer) that is added, is 1:10 to 1:250.
(B) The macromonomer indicated by Formula (1) above is an acrylic acid derivative or a methacrylic acid derivative having a polyethylene glycol group with 8 to 200 repeat units, the acrylate derivative monomer indicated by Formula (2) above is an acrylic acid derivative or a methacrylic acid derivative having a substituent group including an alkyl group having 1 to 12 carbon atoms, and the acrylamide derivative monomer indicated by Formula (4) above is an acrylamide derivative or a methacrylamide derivative having a substituent group including an alkyl group having 1 to 18 carbon atoms.
(C) The polymerization solvent is a mixed solvent containing water and an alcohol, the alcohol being one or more alcohols selected from among ethanol, dipropylene glycol, 1,3-butylene glycol and isoprene glycol.
(E) The solvent composition of the mixed solvent of water and alcohol is such that water:alcohol=90 to 10:10 to 90, in terms of the mass ratio at 20° C.

Hereinafter, the respective conditions will be explained in further detail.

### (Condition (A))

The added molar amount of the polyethylene oxide macromonomer and the hydrophobic monomer (i.e., the sum of the acrylate derivative monomer and/or the acrylamide derivative monomer) is such that polymerization is possible within a range such that polyethylene oxide macromonomer:hydrophobic monomer=1:10 to 1:250 (molar ratio). The added molar amount is preferably 1:10 to 1:200, more preferably 1:25 to 1:100.

If the molar amount of the hydrophobic monomer is less than 10 times the molar amount of the polyethylene oxide macromonomer, then the polymerized polymer becomes water-soluble and core-corona particles are not formed. Additionally, if the molar amount of the hydrophobic monomer is more than 250 times the molar amount of the polyethylene oxide macromonomer, then the dispersion stabilization due to the polyethylene oxide macromonomer becomes incomplete, and hydrophobic polymers formed by the insoluble hydrophobic monomer aggregate and precipitate.

### (Condition (B))

Condition (B) consists of the three conditions (B-1) to (B-3) indicated below.

### (B-1)

The macromonomer represented by Formula (1) is an acrylic acid derivative or a methacrylic acid derivative having a polyethylene glycol group with 8 to 200 repeat units. If there are 7 or fewer repeat units, then there are cases in which particles that are stably dispersed in the solvent cannot be obtained. If there are more than 200, then the particles become small and there are cases in which they become unstable when blended in a cosmetic.

### (B-2)

The acrylate derivative monomer indicated by Formula (2) above is an acrylic acid derivative or a methacrylic acid derivative having a substituent group including an alkyl group having 1 to 12 carbon atoms. If the number of carbon atoms is 0 (if the monomer has no terminal ester bonds), then there are cases in which the monomer is too hydrophilic and good emulsion polymerization cannot be achieved. On the other hand, if the number of carbon atoms is 13 or more, then there are cases in which a favorable feeling in use cannot be obtained.

### (B-3)

The acrylamide derivative monomer indicated by Formula (4) above is an acrylamide derivative or a methacrylamide derivative having a substituent group including an alkyl group having 1 to 18 carbon atoms.

The hydrophobic monomer according to the present invention must be a monomer composition containing one type or a mixture of two or more types of the acrylate derivative monomers represented by Formula (2) above and the acrylamide derivative monomers represented by Formula (4).

In the present invention, the hydrophobic monomer may preferably be of two types, namely methacrylate and butyl methacrylate, or may preferably be of four types, namely, methacrylate, t-butyl acrylamide, N,N-dimethylacrylamide and N-[3-(dimethylamino)propyl] acrylamide. With these combinations of hydrophobic monomers, it is more preferable to use a methoxy polyethylene glycol monomethalate as the macromonomer.

Examples of the most preferable combinations of macromonomers and hydrophobic monomers in the present invention include, but are not limited to:
methoxy polyethylene glycol monomethalates, methacrylates and butyl methacrylates having 8 to 90, most preferably 15 repeat units in the polyethylene glycol group; and
methoxy polyethylene glycol monomethalates, methacrylates, t-butyl acrylamides and N,N-dimethylacrylamides having 8 to 200, most preferably 90 repeat units in the polyethylene glycol group, and N-[3-(dimethylamino)propyl] acrylamide, t-butyl methacrylamide, octyl acrylamide, octyl methacrylamide and octadecyl acrylamide.

### (Condition (C))

The polymerization solvent must be a mixed solvent containing water and an alcohol. The alcohol is preferably one that can dissolve the hydrophobic monomers indicated by Formulas (2) and (4). Thus, one or more alcohols selected from among ethanol, dipropylene glycol, 1,3-butylene glycol and isoprene glycol is preferable.

### (Condition (D))

The solvent composition of the mixed solvent of water and the alcohol, which is the polymerization solvent, should preferably be such that water:alcohol=90 to 10:10 to 90, and more preferably such that water:alcohol=80 to 20:20 to 80, in terms of the mass ratio at 20° C. If the mixing ratio of the alcohol is lower than 10 by volume, then the capacity to dissolve the hydrophobic monomer becomes extremely low, and there are cases in which microparticles are not created. Additionally, if the mixing ratio of the alcohol exceeds 90 by volume, then a hydrophobic monomer emulsion is not formed by hydrophobic interactions, emulsion polymerization does not progress, and microparticles are not obtained.

The microparticles based on conventional synthetic polymers were all obtained by applying polymer electrolytes, for example, polyacrylates, and the dispersion properties thereof in water did not include acid resistance and salt resistance. However, when contemplating application to ingredients in pharmaceutical products and cosmetics, acid resistance and salt resistance are extremely important properties for compatibility with physiological conditions. The core-corona microparticles according to the present invention are microparticles stabilized with polyethylene oxide chains, which are non-ionic polymers. Thus, the dispersion stability thereof in water can be expected to include acid resistance and salt resistance.

In the microparticles used in the present invention, the hydrophilic macromonomer and the hydrophobic monomer become ordered in the solvent, and can be expected to create core-corona polymer microparticles having particles sizes that are approximately uniform and having core moieties that are crosslinked or non-crosslinked.

The blended amounts of the core-corona microparticles of the present invention in the cosmetic are preferably 0.01% to 10% by mass, in terms of pure content, relative to the total amount of the composition. If the blended amount is less than 0.01% by mass (in terms of pure content), then there are cases in which a stable cosmetic becomes difficult to obtain. If the blended amount is more than 10% by mass (in terms of pure content), then there are cases in which the composition is not favorable for the purposes of stability in long-term storage under high-temperature conditions, and cases in which the feeling in use is poor.

### <(B) Self-Tanning Agent>

The (B) self-tanning agent (hereinafter sometimes referred to simply as “component (B)”) blended into the cosmetic according to the present invention refers to a compound, an α-hydroxy aldehyde or a ketone that, when coming into contact with skin, reacts with amino acids and amino groups in skin keratin to form brown compounds. Specific examples include dihydroxyacetone (DHA), 3,4-dihydroxyphenyl pyruvic acid, 3,4-dihydroxyphenyl acetic acid, 3,4-dihydroxyphenyl ethanol, 3,4-dihydroxy mandelic acid, those containing 3,4-dihydroxyphenyl ethylene glycol and ferrous salts. In the present invention, dihydroxyacetone (DHA) is preferably used.

The blended amount of component (B) is 0.1% to 15% by mass, preferably 0.5% to 10% by mass and more preferably 1% to 8% by mass relative to the total amount of the cosmetic. If the blended amount of component (B) is less than 0.1% by mass, then the skin will be insufficiently colored, and if more than 15% by mass is blended, then the stability is poor.

The core-corona microparticles in the present invention form a gel with a solvent (such as water), adsorb to the interface, and emulsify the oil phase components with the water phase components. Thus, compositions in which core-corona microparticles are blended as emulsifying agents are oil-in-water compositions having structures in which a core-corona microgel is adsorbed to interfaces with the oil-phase components dispersed in the water-phase components, or water-in-oil compositions having structures in which a core-corona microgel is adsorbed to interfaces with water-phase components dispersed in the oil-phase components. Therefore, the core-corona microgel emulsifying agent of the present invention has excellent emulsion power, and by using the core-corona microparticles of the present invention as an emulsifying agent, an emulsion cosmetic having very excellent emulsion stability can be produced. Furthermore, the core-corona microgel can obtain sufficient strength even against the activity of hydrophobic powders having large specific gravities present in the oil phase.

### [Oil Phase Components]

Examples of oil phase components include hydrocarbon oils, higher fatty acids, higher alcohols, synthetic ester oils, silicone oils, liquid oils/fats, solid oils/fats, waxes, ultraviolet protectants, oil phase thickeners, hydrophobic powders, fragrances and the like, which are normally used in cosmetics.

Hydrocarbon oils include, for example, isododecane, isohexadecane, isoparaffin, liquid paraffin, ozokerite, squalane, pristane, paraffin, ceresin, squalene, vaseline, microcrystalline wax and the like.

Higher fatty acids are fatty acids having 6 or more carbon atoms, including, for example, lauric acid, myristic acid, palmitic acid, stearic acid, behenic acid, oleic acid, undecylenic acid, tall oil acid, isostearic acid, linolic acid, linoleic acid, eicosapentaenoic acid (EPA), docosahexaenoic acid (DHA) and the like.

Higher alcohols are alcohols having 12 or more carbon atoms, including, for example, linear alcohols (for example, lauryl alcohol, cetyl alcohol, stearyl alcohol, behenyl alcohol, myristyl alcohol, oleyl alcohol, cetostearyl alcohol, etc.), branched alcohols (for example, monostearyl glycerin ether (batyl alcohol)-2-decyl tetradecynol, lanolin alcohol, cholesterol, phytosterol, hexyl dodecanol, isostearyl alcohol, octyl dodecanol, etc.) and the like.

Synthetic ester oils include, for example, isopropyl myristate, cetyl ethylhexanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprate, diisostearyl malate, glycerin di-2-heptylundecanoate, trimethylolpropane tri-2-ethylhexanoate, trimethylolpropane triisostearate, glycerin triisostearate, pentaerythrityl tetraethylhexanoate, triethylhexanoin (glyceryl tri-2-ethylhexanoate), cetyl 2-ethylhexanoate, 2-ethylhexyl palmitate, glycerin trimyristate, glyceride tri-2-heptylundecanoate, castor oil fatty acid methyl ester, oleyl oleate, cetostearyl alcohol, acetoglyceride, 2-heptylundecyl palmitate, diisobutyl adipate, N-lauroyl-L-glutamic acid 2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, polypropylene glycol dipivalate, ethyl acetate, butyl acetate, amyl acetate, triethyl citrate and the like.

Silicone oils include, for example, chain polysiloxanes (such as, for example, dimethyl polysiloxane, methyl phenyl polysiloxane, diphenyl polysiloxane, etc.), cyclic polysiloxanes (for example, octamethyl cyclotetrasiloxane, decamethyl cyclopentasiloxane, dodecamethyl cyclohexasiloxane, etc.), silicone resins that form a three-dimensional mesh structure, silicone rubber, various types of modified polysiloxanes (amino-modified polysiloxane, polyether-modified polysiloxane, alkyl-modified polysiloxane, fluorine-modified polysiloxane, etc.), acryl silicones and the like.

Liquid oils/fats include, for example, avocado oil, camellia oil, turtle oil, macadamia nut oil, corn oil, mink oil, olive oil, rapeseed oil, egg yolk oil, sesame oil, persic oil, wheat germ oil, sasanqua oil, castor oil, linseed oil, safflower oil, cottonseed oil, perilla oil, soybean oil, peanut oil, tea seed oil, Japanese torreya seed oil, rice bran oil, Paulownia fargesii oil, Paulownia tomentosa oil, jojoba oil, germ oil, triglycerin and the like.

Solid oils/fats include, for example, cacao butter, coconut oil, horse fat, hardened coconut oil, palm oil, beef tallow, mutton tallow, hardened beef tallow, palm kernel oil, lard, beef bone fat, Toxicodendron succedaneum kernel oil, hardened oil, neatsfoot oil, Japan wax, hardened castor oil and the like.

Waxes include, for example, beeswax, candelilla wax, cotton wax, carnauba wax, bayberry wax, insect wax, spermaceti, montan wax, rice bran wax, lanolin, kapok wax, lanolin acetate, liquid lanolin, sugarcane wax, lanolin fatty acid isopropyl ester, hexyl laurate, reduced lanolin, jojoba wax, hardened lanolin, shellac wax, POE lanolin alcohol ether, POE lanolin alcohol acetate, POE cholesterol ether, lanolin fatty acid polyethylene glycol, POE hydrogenated lanolin alcohol ether and the like.

In emulsion cosmetics emulsified with conventional surfactants, the physical properties of the surfactant and the physical properties of the oil component largely affected the emulsification properties. Thus, when the oil phase components were changed, there was a need to respond by changing the types of surfactants and the like. However, the emulsion cosmetic of the present invention is a Pickering emulsion having core-corona microparticles as a dispersant. Therefore, the type of oil component has little influence on the emulsion properties and the stability, and a wider range of types of oil components can be added than in conventional emulsion cosmetics.

In the self-tanning emulsion cosmetic of the present invention, the effects of protecting the skin from ultraviolet rays can be imparted by further blending a (C) ultraviolet protectant (hereinafter sometimes referred to simply as “component (C)”).

The ultraviolet protectant blended in the cosmetic of the present invention refers to an ultraviolet absorbing agent and/or an ultraviolet scattering agent, and one that is normally blended in cosmetics may be used.

The ultraviolet absorbing agents that can be used in the present invention are not particularly limited, and a wide range of ultraviolet absorbing agents that are generally used in cosmetics can be mentioned. Examples include benzoic acid derivatives, salicylic acid derivatives, cinnamic acid derivatives, dibenzoyl methane derivatives, β,β-diphenyl acrylate derivatives, benzophenone derivatives, benzylidene camphor derivatives, phenylbenzimidazole derivatives, triazine derivatives, phenylbenzotriazole derivatives, anthranil derivatives, imidazoline derivatives, benzalmalonate derivatives, 4,4-diaryl butadiene derivatives and the like. Hereinafter, specific examples and product names will be mentioned, but there is no limitation thereto.

Examples of benzoic acid derivatives include ethyl para-aminobenzoate (PABA), ethyl-dihydroxypropyl PABA, ethylhexyl-dimethyl PABA (e.g., “Escalol 507”; ISP), glyceryl PABA, PEG-25-PABA (e.g., “Uvinul P25”; BASF), diethylamino hydroxybenzoyl hexyl benzoate (e.g., “Uvinul A Plus”) and the like.

Examples of salicylic acid derivatives include homosalate (“Eusolex HMS”; Rona/EM Industries), ethylhexyl salicylate (e.g., “Neo Heliopan OS”; Haarmann & Reimer), dipropylene glycol salicylate (e.g., “Dipsal”; Scher), TEA salicylate (e.g., “Neo Heliopan TS”; Haarmann & Reimer) and the like.

Examples of cinnamic acid derivatives include octyl methoxycinnamate or ethylhexyl methoxycinnamate (e.g., “Parsol MCX”; Hoffmann-La Roche), isopropyl methoxycinnamate, isoamyl methoxycinnamate (e.g., “Neo Heliopan E1000”; Haarmaan & Reimer), cinnoxate, DEA methoxycinnamate, diisopropyl methyl cinnamate, glyceryl ethylhexanoate dimethoxycinnamate, di-(2-ethylhexyl)-4′-methoxybenzalmalonate and the like.

Examples of dibenzoyl methane derivatives include 4-tert-butyl-4′-methoxy dibenzoyl methane (e.g., “Parsol 1789”) and the like.

Examples of β,β-diphenyl acrylate derivatives include octocrylene (e.g., “Uvinul N539T”; BASF) and the like.

Examples of benzophenone derivatives include benzophenone-1 (e.g., “Uvinul 400”; BASF), benzophenone-2 (e.g., “Uvinul D50”; BASF), benzophenone-3 or oxybenzone (e.g. “Uvinul M40”; BASF), benzophenone-4 (e.g., “Uvinul MS40”; BASF), benzophenone-5, benzophenone-6 (e.g., “Helisorb 11”; Norquay), benzophenone-8 (e.g., “Spectra-Sorb UV-24”; American Cyanamid), benzophenone-9 (e.g., “Uvinul DS-49”; BASF), benzophenone-12 and the like.

Examples of benzylidene camphor derivatives include 3-benzylidene camphor (e.g., “Mexoryl SD”; Chimex), 4-methylbenzylidene camphor, benzylidene camphor sulfonic acid (e.g., “Mexoryl SL”; Chimex), camphor benzalkonium methosulfate (e.g., “Mexoryl SO”; Chimex), terephthalylidene dicamphor sulfonic acid (e.g., “Mexoryl SX”; Chimex), polyacrylamide methylbenzylidene camphor (e.g., “Mexoryl SW”; Chimex) and the like.

Examples of phenylbenzimidazole derivatives include phenylbenzimidazole sulfonic acid (e.g., “Eusolex 232”; Merck), disodium phenyldibenzimidazole tetrasulfonate (e.g., “Neo Heliopan AP”; Haarmann & Reimer) and the like.

Examples of triazine derivatives include bis-ethylhexyloxyphenol methoxyphenyl triazine (e.g., “Tinosorb S”; Ciba Specialty Chemicals), ethylhexyl triazone (e.g., “Uvinul T150”; BASF), diethylhexyl butamido triazone (e.g., “Uvasorb HEB”; Sigma 3V), 2,4,6-tris(diisobutyl-4′-aminobenzalmalonate)-s-triazine, 2,4,6-tris[4-(2-ethylhexyloxycarbonyl)anilino]-1,3,5-triazine and the like.

Examples of phenylbenzotriazole derivatives include drometrizole trisiloxane (e.g., “Silatrizole”; Rhodia Chimie), methylene bis(benzotriazolyl tetramethylbutyl phenol) (e.g., “Tinosorb M”; Ciba Specialty Chemicals) and the like.

Examples of anthranil derivatives include menthyl anthranilate (e.g., “Neo Heliopan MA”; Haarmann & Reimer) and the like.

Examples of imidazoline derivatives include ethylhexyl dimethoxybenzylidene dioxoimidazoline propionate and the like.

Examples of benzalmalonate derivatives include polyorganosiloxanes having benzalmalonate functional groups (e.g., Polysilicone-15; “Parsol SLX”; DSM Nutrition Japan) and the like.

Examples of 4,4-diarylbutadiene derivatives include 1,1-dicarboxy (2,2′-dimethylpropyl)-4,4-diphenylbutadiene and the like.

Particularly preferred examples include, but are not limited to, ethylhexyl methoxycinnamate, octocrylene, dimethicodiethyl benzalmalonate, polysilicone-15, 4-tert-butyl-4′-methoxy dibenzoyl methane (t-butyl methoxy dibenzoyl methane), ethylhexyl triazone, diethylamino hydroxybenzoyl hexyl benzoate, bis-ethylhexyloxyphenol methoxyphenyl triazine, oxybenzone-3, methylene bisbenzotriazolyl tetramethylbutyl phenol, phenylbenzimidazole sulfonic acid, 3-(4′-methylbenzylidene)-d,l-camphor, 3-benzylidene-d,l-camphor, homosalate, ethylhexyl salicylate and the like. The ultraviolet absorbing agent used in the present invention may be blended as one type or as a combination of two or more types.

The ultraviolet scattering agent used in the present invention is not particularly limited, but specific examples include fine-particle metal oxides such as, for example, zinc oxide, titanium oxide, iron oxide, cerium oxide and tungsten oxide.

The ultraviolet scattering agent may be non-surface-treated or may be treated with various types of hydrophobic surface treatments, but those that are hydrophobically surface-treated are preferably used. As the surface treatment agent, it is possible to use a type that is commonly used in the cosmetics field including, for example, a silicone such as dimethicone or alkyl-modified silicone, an alkoxysilane such as octyltriethoxysilane, a dextrin fatty acid ester such as dextrin palmitate, or a fatty acid such as stearic acid.

The ultraviolet protectant in the present invention includes embodiments consisting only of an ultraviolet absorbing agent, embodiments consisting only of an ultraviolet scattering agent, and embodiments containing both an ultraviolet absorbing agent and an ultraviolet scattering agent.

Although the blended amount of the ultraviolet protectant is not particularly limited, the amount should normally be at least 5% by mass, for example, 5% to 40% by mass, preferably 6% to 40% by mass, and more preferably 7% to 35% by mass relative to the total amount of the emulsion cosmetic. If the blended amount of the ultraviolet protectant is less than 5% by mass, then sufficient ultraviolet protection effects are difficult to obtain, and even if more than 40% by mass is blended, an increase in the ultraviolet protection effects commensurate with the blended amount cannot be expected, and the stability is worsened.

In particular, when blending an ultraviolet scattering agent, the blended amount should preferably be 5% by mass or less, more preferably 0% to 2% by mass or less, relative to the total amount of the cosmetic, for the purposes of suppressing whitening after application.

As oil phase thickeners, substances that are used, in emulsion cosmetics and the like, as components for obtaining the effect of thickening the oil phase by dissolving into oils or being swollen by oils are preferable. Examples include dextrin fatty acid esters such as dextrin palmitate and dextrin myristate, sucrose fatty acid esters such as sucrose caprylic acid ester, solid or semi-solid hydrocarbon oils such as vaseline, hydrogenated palm oil and hydrogenated castor oil, organically modified clay minerals such as disteardimonium hectorite and benzyl dimethyl stearyl ammonium hectorite, or higher fatty acids having 8 to 22 carbon atoms that are solid at ambient temperature, such as lauric acid, myristic acid, palmitic acid and stearic acid or salts thereof, and the like.

In the emulsion cosmetic according to the present invention, a hydrophobic powder may be blended into the oil phase. According to the present invention, the stability can be improved without gelling by means of large amounts of surfactants or thickening by means of polymer substances. Thus, the water resistance of the hydrophobic powder can be sufficiently obtained.

In the present invention, there is a tendency for the water resistance and the rubbing resistance to further improve by blending the core-corona microparticles and the hydrophobic powder together.

The hydrophobic powder is not particularly limited as long as the surface of the powder is hydrophobic, but examples include powders in which the powders themselves are hydrophobic, such as silicone resin powders and fluorine resin powders, and powders obtained by hydrophobically treating the surfaces of inorganic powder particles, by means of wet methods using solvents, vapor phase methods or mechanochemical methods, using silicones such as methylhydrogen polysiloxane and dimethyl polysiloxane, or hydrocarbons such as dextrin fatty acid esters, higher fatty acids, higher alcohols, fatty acid esters, metal soaps, alkyl phosphate ethers, fluorine compounds, or squalane or paraffin. The average particle size of the hydrophobic powder must be smaller than that of the emulsion particles constituting the oil phase in the present invention. In particular, when using a powder as an ultraviolet scattering agent, it is preferable to use one having an average particle size of 100 nm or smaller after being crushed in a wet disperser. Examples of inorganic powder particles that are hydrophobically treated include titanium oxide, zinc oxide, talc, mica, sericite, kaolin, titanated mica, black iron oxide, yellow iron oxide, red iron oxide, ultramarine blue, Prussian blue, chromium oxide, chromium hydroxide and the like.

Examples of fragrances include, but are not particularly limited to, natural fragrances obtained from animals or plants, synthetic fragrances manufactured by chemical synthesis means, and blended fragrances, which are mixtures thereof. By blending a fragrance, a cosmetic having an exceptionally long-lasting aroma can be obtained.

### [Water Phase Components]

As water phase components, it is possible to blend water, lower alcohols, polyhydric alcohols, water-soluble polymers and the like that are normally used in cosmetics. Furthermore, a humectant, a powder component or the like may be appropriately blended as needed.

The water contained in the emulsion cosmetic of the present invention is not particularly limited, and examples include purified water, ion-exchanged water, tap water and the like.

Lower alcohols include, for example, alcohols having 1 to 5 carbon atoms such as ethanol, propanol, isopropanol, isobutyl alcohol and t-butyl alcohol.

Polyhydric alcohols include, for example, dihydric alcohols (for example, dipropylene glycol, 1,3-butylene glycol, ethylene glycol, trimethylene glycol, 1,2-butylene glycol, tetramethylene glycol, 2,3-butylene glycol, pentamethylene glycol, 2-butene-1,4-diol, hexylene glycol, octylene glycol, etc.), trihydric alcohols (for example, glycerin, trimethylolpropane, etc.), tetrahydric alcohols (for example, diglycerin, pentaerythritols such as 1,2,6-hexanetriol, etc.), pentahydric alcohols (for example, xylitol, triglycerin, etc.), hexahydric alcohols (for example, sorbitol, mannitol, etc.), polyhydric alcohol polymers (for example, diethylene glycol, dipropylene glycol, triethylene glycol, polypropylene glycol, tetraethylene glycol, diglycerin, triglycerin, tetraglycerin, polyglycerin, etc.), dihydric alcohol alkyl ethers (for example, ethylene glycol monomethyl ether, ethylene glycol monoethyl ether, ethylene glycol monobutyl ether, ethylene glycol monophenyl ether, ethylene glycol monohexyl ether, ethylene glycol mono-2-methylhexyl ether, ethylene glycol isoamyl ether, ethylene glycol benzyl ether, ethylene glycol isopropyl ether, ethylene glycol dimethyl ether, ethylene glycol diethyl ether, ethylene glycol butyl ether, etc.), dihydric alcohol alkyl ethers (for example, diethylene glycol monomethyl ether, diethylene glycol monoethyl ether, diethylene glycol monobutyl ether, diethylene glycol dimethyl ether, diethylene glycol diethyl ether, diethylene glycol butyl ether, diethylene glycol methylethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether, etc.), dihydric alcohol ether esters (for example, ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disuccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate, etc.), glycerin monoalkyl ethers (for example, xyl alcohol, selachyl alcohol, batyl alcohol, etc.), sugar alcohols (for example, maltotriose, mannitol, sucrose, erythritol, glucose, fructose, starch-decomposed sugars, maltose, starch-decomposed sugar-reduced alcohols, etc.), glysolid, tetrahydrofurfuryl alcohol, POE-tetrahydrofurfuryl alcohol, POP-butyl ether, POP/POE-butyl ether tripolyoxypropylene glycerin ether, POP-glycerin ether, POP-glycerin ether phosphoric acid, POP/POE-pentane erythritol ether, polyglycerin and the like.

Water-soluble polymers include homopolymers or copolymers of 2-acrylamido-2-methylpropane sulfonic acid (hereinafter abbreviated to “AMPS”). The copolymers are copolymers comprising comonomers such as vinyl pyrrolidone, acrylic acid amides, sodium acrylate and hydroxyethyl acrylate. In other words, examples include AMPS homopolymers, vinyl pyrrolidone/AMPS copolymers, dimethylacrylamide/AMPS copolymers (for example, (dimethylacrylamide/sodium acryloyldimethyl taurate) copolymer), acrylic acid amide/AMPS copolymers, sodium acrylate/AMPS copolymers and the like. In the cosmetic of the present invention, (dimethylacrylamide/sodium acryloyldimethyl taurate) copolymer is preferably used.

Further examples include carboxyvinyl polymers, ammonium polyacrylates, sodium polyacrylates, sodium acrylate/alkyl acrylate/sodium methacrylate/alkyl methacrylate copolymers, carrageenan, pectin, mannan, curdlan, chondroitin sulfate, starch, glycogen, gum arabic, sodium hyaluronate, tragacanth gum, xanthan gum, mucoitin sulfate, hydroxyethyl guar gum, carboxymethyl guar gum, guar gum, dextran, keratosulfate, locust bean gum, succcinoglucan, chitin, chitosan, carboxymethyl chitin, agar and the like.

Humectants include, for example, trehalose, chondroitin sulfate, hyaluronic acid, mucoitin sulfate, caronic acid, atelocollagen, cholesteryl-12-hydroxystearate, sodium lactate, bile acid salts, DL-pyrrolidone carboxylic acid salts, short-chain soluble collagens, diglycerin (EO)PO adduct, Rosa roxburghii extract, Achillea millefolium extract, melilot extract and the like.

Powder components include, for example, inorganic powders (for example, silica, talc, kaolin, mica, sericite, white mica, gold mica, synthetic mica, red mica, black mica, vermiculite, magnesium carbonate, calcium carbonate, aluminum silicate, barium silicate, calcium silicate, magnesium silicate, strontium silicate, tungstic acid metal salts, magnesium, zeolite, barium sulfate, sintered calcium sulfate (burnt plaster), calcium phosphate, fluorapatite, hydroxyapatite, ceramic powder, metal soaps (for example, zinc myristate, calcium palmitate and aluminum stearate), boron nitride, etc.), organic powders (for example, polyamide resin powders (nylon powder), polyethylene powders, polymethyl methacrylate powders, polystyrene powders, styrene-acrylic acid copolymer resin powders, benzoguanamine resin powders, polytetrafluoroethylene powders, cellulose powders, etc.), inorganic white pigments (for example, titanium oxide, zinc oxide, etc.), inorganic red pigments (for example, iron oxide (red iron oxide), iron titanate, etc.), inorganic brown pigments (for example, y-iron oxide), inorganic yellow pigments (for example, yellow iron oxide, ocher, etc.), inorganic black pigments (for example, black iron oxide, low-order titanium oxide, etc.), inorganic violet pigments (for example, mango violet, cobalt violet, etc.), inorganic green pigments (for example, chromium oxide, chromium hydroxide, cobalt titanate, etc.), inorganic blue pigments (for example, ultramarine blue, Prussian blue, etc.); pearlescent pigments (for example, titanium oxide-coated mica, titanium oxide-coated bismuth oxychloride, titanium oxide-coated talc, colored titanium oxide-coated mica, bismuth oxychloride, argentine, etc.), metal powder pigments (for example, aluminum powder, copper powder, etc.), organic pigments such as zirconium, barium or aluminum lakes (for example, organic pigments such as Red No. 201, Red No. 202, Red No. 204, Red No. 205, Red No. 220, Red No. 226, Red No. 228, Red No. 405, Orange No. 203, Orange No. 204, Yellow No. 205, Yellow No. 401 and Blue No. 404; Red No. 3, Red No. 104, Red No. 106, Red No. 227, Red No. 230, Red No. 401, Red No. 505, Orange No. 205, Yellow No. 4, Yellow No. 5, Yellow No. 202, Yellow No. 203, Green No. 3, Blue No. 1, etc.), natural pigments (for example, chlorophyll, 13-carotene, etc.) and the like.

### [Other Components]

In the emulsion cosmetic of the present invention, an anionic surfactant, a cationic surfactant, an amphoteric surfactant, a non-ionic surfactant or the like may be blended, as appropriate, in accordance with the format, such as oil-in-water or water-in-oil. For example, in the case of an oil-in-water emulsion cosmetic, a non-ionic surfactant having an HLB of 6 or higher, such as PEG-10 hydrogenated castor oil, PEG-30 hydrogenated castor oil, PEG-50 hydrogenated castor oil, PEG-60 hydrogenated castor oil or PEG-100 hydrogenated castor oil is preferably blended. Additionally, in the case of a water-in-oil emulsion cosmetic, a surfactant having an HLB lower than 8, such as a polyether-modified silicone, a polyether/alkyl-comodified silicone (for example, lauryl PEG-9 polydimethyl polysiloxyethyl dimethicone), a polyglycerin-modified silicone or a polyglycerin/alkyl-comodified silicone is preferably blended.

In the emulsion cosmetic of the present invention, a stable emulsion can be obtained even if the blended amount of the surfactant is low. Thus, the present invention has the effect of providing an excellent texture to the touch. The blended amount of the surfactant relative to the total amount of the cosmetic should preferably be less than 1.5% by mass, more preferably 1.0% by mass or lower, and even more preferably 0.5% by mass or lower.

In the emulsion cosmetic of the present invention, other components that are normally used in cosmetics, for example, neutralizing agents, chelating agents, pH adjusting agents, vitamins, anti-oxidants, preservatives and the like may be appropriately blended within a range not compromising the effects of the present invention.

The emulsion cosmetic of the present invention may be formulated in either oil-in-water form or in water-in-oil form. However, it is more preferably prepared as an oil-in-water emulsion cosmetic for the purpose of further maintaining long-term stability of the self-tanning agent.

The emulsion cosmetic of the present invention is produced by a conventional method such as by mixing and dispersing the core-corona microparticles in water or in the water phase components, adding the oil phase components and other components, and emulsifying by stirring and applying a shearing force.

The blended amounts of the oil phase components and the water phase components blended in the powder-in oil-in water composition of the present invention are not particularly limited. By using (a) core-corona microparticles as the emulsifying agent, emulsion cosmetics in a wide range of formats, from embodiments (such as gels and foams) with low ratio of the oil phase components to the water phase components, i.e., low blended amounts of oil phase components to embodiments (such as creams) with high blended amounts thereof, can be obtained.

### EXAMPLES

Hereinafter, the present invention will be explained in further detail by providing examples. However, these examples do not limit the present invention in any way. Where not specially indicated otherwise, the blended amounts are indicated in percentage by mass relative to the system in which the relevant component is blended.

### 1. Production of Non-Crosslinked Core-Corona Microparticle Dispersion

The macromonomers and hydrophobic monomers indicated in Table 1 were radical-polymerized in accordance with the production method (process 1) indicated below, under the polymerization conditions indicated in Table 1 and Table 2. The appearance of the obtained copolymer dispersion was evaluated by visual observation, and the particle size and the degree of dispersion of the copolymer were evaluated in accordance with process 2.

### <Process 1: Method for Producing Non-Crosslinked Core-Corona Microparticle Dispersion>

The polyethylene oxide macromonomer and the hydrophobic monomer were added to 90 g of a mixed water-alcohol solvent in a three-necked flask equipped with a reflux tube and a nitrogen-feeding tube. After the monomers were well dissolved or dispersed, the solution was purged with nitrogen for 20 minutes to remove the dissolved oxygen. To this solution, 1 mol %, relative to the total monomer amount, of polymerization initiator, 2,2′-azobis(2-methylpropionamidine dihydrochloride) was added by being dissolved in a small amount of water, and further dissolved or dispersed. The homogeneously dissolved or dispersed polymer solution was purged with nitrogen for 20 minutes to remove the dissolved oxygen, after which a polymerization reaction was induced by keeping the solution for 8 hours in an oil bath at 65 to 75° C. while stirring with a magnetic stirrer. After the polymerization ended, the polymer solution was returned to room temperature to obtain the core-corona microparticle dispersion.

In Table 1 below, Blemmer PME-4000 (manufactured by NOF) was used as the polyethylene oxide macromonomer, and methyl methacrylate (MMA), butyl methacrylate (n-BMA), t-butyl acrylamide (t-BAA) or N-[3-(dimethylamino)propyl] acrylamide (DMAPA) was used as the hydrophobic monomer. The units of the numerical values in Table 1 are all in g (grams).

TABLE 1

| Macro- | | | | | | | |
|---|---|---|---|---|---|---|---|
| monomer | | | | | | | |
| Methoxy PEG | Hydrophobic Monomer | | | | Polymerization Solvent | | |
| 4000 | MMA | n-BMA | t-BAA | DMAPA | | alcohol | alcohol |
| Formula (1) | Formula (2) | Formula (2) | Formula (4) | Formula (4) | water | type | amount |
| 4.06 | 2.40 | 3.41 | 0.06 | 0.08 | 54 | ethanol | 36 |

TABLE 2

| (A) Macro- | | | | | | | (D) |
|---|---|---|---|---|---|---|---|
| monomer/ | | | | | | | Water/ |
| Hydrophobic | (B-1) | | | | | | alcohol |
| monomer | Macro- | (B-2) Acrylate | | (B-3) Acrylamide derivative | | (C) | solvent |
| ratio | monomer | derivative monomer | | monomer | | Alcohol | mixing |
| (molar ratio) | Formula (1) | Formula (2) | | Formula (4) | | type | ratio |
| 1/50 | R₁ = CH₃ | R₂ = CH₃ | R₂ = CH₃ | R₄ = H | R₄ = H | ethanol | 60/40 |
| | n = 90 | R₃ = CH₃ | R₃ = nC₄H₉ | R₅ = H | R₅ = H | | |
| | | | | R₆ = tC₄H₉ | R₆ = C₃H₆N(CH₃)₂ | | |

### <Process 2: Method for Measuring Particle Size and Degree Of Dispersion>

The particle size of the copolymer was measured by using a Zetasizer manufactured by Malvern. A measurement sample having a microparticle concentration of approximately 0.1% was prepared from the microparticle dispersion by means of water dilution. After removing impurities with a 0.45 micrometer filter, the scattering intensity at 25° C. was measured at a scattering angle of 173° (back-scattered light), and the average particle size and degree of dispersion were computed with analysis software installed in the measurement device. The particle size was analyzed by means of the cumulant method, and the degree of dispersion was the numerical value obtained by normalizing the value of the second-order cumulant obtained by cumulant analysis. This degree of dispersion is a generally used parameter, which can be automatically analyzed by using a commercially available dynamic light scattering measurement device. As the viscosity of the solvent necessary for the particle size analysis, the viscosity of pure water at 25° C., i.e., 0.89 mPa·s, was used.

The appearance of the resulting copolymer dispersion was that of a cloudy white liquid. Additionally, the core-corona microparticle concentration was 10% by weight, the alcohol type and the alcohol concentration were ethanol and 36% by weight, and the water concentration was 90% by weight. The average particle size of the copolymer dispersion was 210.3 nm and the degree of dispersion was 0.018.

### 2. Production of Emulsion Cosmetic

Next, the core-corona microparticles produced as described above were used to produce cosmetics with the formulations indicated in Table 3. For each cosmetic, the oil phase components among the components indicated in the table were heated and mixed homogeneously to prepare an oil phase portion, and the powder components were mixed into this oil phase portion to obtain a mixture. Next, the water phase components were heated and dissolved to prepare a water phase portion, added to the mixture, and emulsified by a stirring process to produce oil-in-water emulsion cosmetics (Formulation Examples 1 to 3) and a water-in-oil emulsion cosmetic (Formulation Example 4).

### 3. Cosmetic Evaluation Method

The prepared cosmetics were evaluated regarding preparation stability, water resistance, rubbing resistance and texture (lack of stickiness, wateriness) in accordance with the evaluation methods indicated below. The evaluation results are indicated in Table 3.

### Evaluation 1: Preparation Stability

Regarding the stability over time of the prepared cosmetics, after one month at rest at 50° C., the stability was evaluated by visual observation based on the standards below.

A: Uniformly emulsified
B: Some floating oil observed
C: Separation observed

### Evaluation 2: Water Resistance

For the water resistance, the ultraviolet protection performance of the ultraviolet absorbing agents blended into the cosmetics were measured before and after being bathed in water, and the fraction of the ultraviolet protection performance remaining after being bathed in water (the absorbance survival rate) was computed to measure the strength of the water resistance. Specifically, cosmetics (samples) according to each example were dripped, at a rate of 2 mg/cm², onto measurement plates (S plates) (5×5 cm V-groove PMMA plates, SPFMASTER-PA01), applied by finger for 60 seconds, and dried for 15 minutes to form coating films, the absorbances of which were measured using a Hitachi U-3500 self-recording spectrophotometer. The absorbances (Abs) were computed, with glycerin, which does not absorb ultraviolet rays, as the control, by using the following equation.

Abs=−log (T/To)

T: transmittance of sample, To: transmittance of glycerin

The measured plates were fully immersed in water having a hardness of 50 to 500, and stirred for 30 minutes in water (3-1 motor at 300 rpm). Thereafter, the plates were dried for approximately 15 to 30 minutes until droplets on the surfaces disappeared, the absorbances were measured again, and the Abs survival rates (the equation below) were computed from the Abs integral values before and after being bathed in water. Abs survival rate (%)=(Abs integral value after being bathed in water)/(Abs integral value before being bathed in water)×100

Evaluations were made under the standards below based on the computed Abs survival rates.

A: at least 70% survival
B: at least 50% and less than 70% survival
C: less than 50% survival

### Evaluation 3: Rubbing Resistance

For the rubbing resistance, the ultraviolet protection performance of the ultraviolet absorbing agents blended into the cosmetics were measured before and after rubbing tests, and the fraction of the ultraviolet protection performance remaining after the rubbing tests (the absorbance survival rate) was computed to measure the strength of the rubbing resistance. Specifically, samples of each example were dripped, at a rate of 2 mg/cm², onto S plates (5 ×5 cm V-groove PMMA plates, SPFMASTER-PA01), applied by finger for 60 seconds and dried for 15 minutes, then the absorbances thereof (at 400 to 280 nm) were measured using a Hitachi U-3500 self-recording spectrophotometer. The absorbances (Abs) were computed, with uncoated plates as the control, by using the following equation.

Abs=−log (T/To)

T: transmittance of sample, To: transmittance of uncoated plate

Next, the measured plates were placed with the coated surfaces of the S plates facing upward, and the plates were rubbed 10 times with uniform pressure, using fingers wrapped in tissue paper. Thereafter, the absorbances of the S plates were measured again with the spectrophotometer.

The Abs survival rates with respect to rubbing were determined from the equation below from the Abs integral values immediately after applying the cosmetics and after being rubbed.

Abs survival rate (%)=(Abs integral value after being rubbed with tissue)/(Abs integral value immediately after application)×100

Evaluations were made under the standards below based on the computed Abs survival rates.

A: at least 80% survival
B: at least 70% and less than 80% survival
C: less than 70% survival

### Evaluation 4: Texture (Lack of Stickiness, Wateriness)

Evaluations were made by having ten expert panelists performing tests of actual use. Specifically, the texture to the touch (lack of stickiness, wateriness) when the prepared samples were applied to skin was evaluated in accordance with the standards indicated below.

### (Lack of Stickiness)

A: Not sticky
B: Somewhat sticky

### C: Sticky

### (Wateriness)

### A: Watery

B: Somewhat watery
C: Not watery

TABLE 3

| | | Form. | Form. | Form. | Form. |
|---|---|---|---|---|---|
| | Component Name | Ex. 1 | Ex. 2 | Ex. 3 | Ex. 4 |
| | Water | bal | bal | bal | bal |
| Core-corona | (Acrylamide/DMAPA acrylate/methoxy PEG | 1 | — | — | — |
| microparticles | methacrylate) copolymer | | | | |
| Tanning component | Dihydroxyacetone | 3 | 3 | 3 | 3 |
| Surfactant | Polyoxyethylene hardened castor oil (60 mol) | 0.3 | 1.5 | 0.1 | — |
| | Lauryl PEG-9 polydimethyl polysiloxyethyl | — | — | — | 3 |
| | dimethicone | | | | |
| | Isostearic acid | — | — | — | 0.5 |
| Water-soluble | (Acrylates/(C10-30) alkyl acrylate) | — | — | 0.1 | — |
| polymer | crosspolymer | | | | |
| | (Dimethylacrylamide/sodium acryloyldimethyl | 0.5 | 0.5 | 0.3 | — |
| | taurate) copolymer | | | | |
| | Succinoglucan | 0.12 | 0.12 | 0.12 | — |
| Alcohol | Ethanol | 8 | 8 | 8 | 8 |
| Humectant | Glycerin | 1 | 1 | 1 | 2 |
| | 1,3-Butylene glycol | 5 | 5 | 5 | — |
| Neutralizing agent | Potassium hydroxide | — | — | 0.05 | — |
| Clay mineral | Dimethyl distearyl ammonium hectorite | — | — | — | 0.5 |
| Oil | Isododecane | 2 | 2 | 2 | 10 |
| | Dimethicone | 5 | 5 | 5 | 8 |
| | Propylene glycol (17) | 1 | 1 | 1 | — |
| | PBG/PPG-9/1 copolymer | — | — | — | 2 |
| | Glyceryl tri-2-ethylhexanoate | — | — | — | 5 |
| | Pentaerythritol tetra-2-ethylhexanoate | — | — | — | 2 |
| | Cetyl ethylhexanoate | — | — | — | 5 |
| | Diisopropyl sebacate | — | — | — | 10 |
| | Carnauba wax | 1 | | 1 | — |
| Ultraviolet | Octocrylene | 7 | 7 | 7 | 7 |
| absorbing agent | Ethylhexyl salicylate | 5 | 5 | 5 | 5 |
| | t-Butyl methoxy dibenzoyl methane | 2.5 | 2.5 | 2.5 | 2.5 |
| | Diethylamino hydroxybenzoyl hexyl benzoate | — | — | — | 2 |
| | Homosalate | 5 | 5 | 5 | — |
| Texture-adjusting | Silica | 1 | 5 | 1 | 1 |
| powder | Methyl methacrylate crosspolymer | — | — | — | 7 |
| Stabilizer | Fragrance/pH adjuster/antioxidant etc. | s.a. | s.a. | s.a. | s.a. |
| | Emulsion type | O/W | O/W | O/W | W/O |
| Evaluation | Preparation stability | A | A | B | A |
| | Water resistance | A | C | B | A |
| | Rubbing resistance | A | C | B | B |
| | Texture (lack of stickiness) | A | C | B | B |
| | Texture (wateriness) | A | B | A | C |

As indicated in Table 3, a cosmetic (Formulation Example 2) that was emulsified by polyoxyethylene hydrogenated castor oil (60 mol), which is a non-ionic surfactant, had poor water resistance and rubbing resistance, and also had an inferior texture. A cosmetic (Formulation Example 3) emulsified by (acrylates/(C10-30) alkyl acrylate) crosspolymer, which is a polymeric surfactant, also had inferior water resistance and rubbing resistance, and had a tendency for stickiness to occur. Additionally, a water-in-oil cosmetic (Formulation Example 4) in which the core-corona microparticles of the present invention were not blended had inferior rubbing resistance, and stickiness occurred.

However, a cosmetic (Formulation Example 1) emulsified by core-corona microparticles exhibited excellent preparation stability, excellent resistance to water and rubbing, and also had a good texture.

1. An emulsion cosmetic for self-tanning, comprising:
(A) core-corona microparticles in which hydrophilic groups are partially provided on surfaces of hydrophobic fine particles; and
(B) a self-tanning agent.

2. The cosmetic according to claim 1, wherein the (B) self-tanning agent is dihydroxyacetone.

3. The cosmetic according to claim 1, further comprising (C) an ultraviolet protectant.

4. The cosmetic according to claim 3, wherein the (C) ultraviolet protectant is an ultraviolet absorbing agent.

5. The cosmetic according to claim 1, wherein a blended amount of an ultraviolet scattering agent is 5% by mass or less.
